(19) United States
(12) Patent Application Publication
(10) Pub. No.: US 2010/0160951 A1
(43) Pub. Date: Jun. 24, 2010

(54) INTRACRANIAL BLOOD VESSEL DILATION DEVICE

(76) Inventor: Michael T. Madison, Edina, MN (US)

Correspondence Address: STOEL RIVES LLP - SLC, 201 SOUTH MAIN STREET, SUITE 1100, ONE UTAH CENTER, SALT LAKE CITY, UT, 84111, US

(21) Appl. No.: 12/340,109

(22) Filed: Dec. 19, 2008

(51) Int. Cl. A61M 29/00

(52) U.S. Cl. 606/198

(57) ABSTRACT

A method of treating cerebral vasospasm includes delivering at least a first blood vessel including an expandable segment to a position within a blocked or constricted portion of a vessel and transitioning the expandable segment from a collapsed configuration to an expanded configuration to dilate the vessel and to restore adequate blood flow through the vessel. The expandable segment has a pre-set outer diameter substantially equal to or less than an inner diameter of the vessel. Additionally, the expandable segment is configured to expand with a minimal amount of radial force necessary to contact and expand the inner walls of the vessel. After dilation of the vessel, the device is removed from the patient's body. If necessary, additional blood vessel dilation devices each including an expandable segment having a pre-set outer diameter greater than the previous device's expandable segment can be used in order of increasing pre-set outer diameter of the expanded segment to further dilate the blocked or constricted portion of the vessel and restore blood flow through the vessel. Once adequate blood flow through the vessel has been restored, the blood vessel dilation device is withdrawn.

### FIELD OF THE INVENTION

The present invention relates to medical devices used during neurovascular procedures. More particularly, the present invention relates to devices and methods for re-establishing a blood flow channel in a blocked or constricted vessel.

### BACKGROUND OF THE INVENTION

A stroke, or cerebrovascular accident (CVA), occurs when blood supply to part of the brain is disrupted, causing brain cells to die. When blood flow to the brain is impaired, oxygen and glucose cannot be delivered to the brain. Blood flow can be compromised by a variety of mechanisms including blockage or constriction of a vessel or vessels supplying blood to the brain. A disruption in blood flow to the brain can result in the loss of neurological function and, in some cases, death.

There is continuing need for devices and methods for re-establishing a channel of blood flow in a vessel that are safe and have a long lasting beneficial effect.

### BRIEF SUMMARY OF THE INVENTION

According to some embodiments, the present invention is a method for dilating a portion of a vessel in which blood flow is restricted to re-establish a blood flow channel including the steps of: selecting at least a first blood vessel dilation device including an elongated microwire having an expandable segment configured to transition from a collapsed configuration to an expanded configuration, the expandable segment comprising at least two struts having a configuration allowing blood flow through the vessel and a pre-set outer diameter substantially equal to or less than an inner diameter of the vessel; advancing a delivery catheter including at least one lumen to a position within the blocked or constricted portion of the vessel; delivering the first blood vessel dilation device through the delivery catheter lumen to the blocked or constricted portion of the vessel, wherein the delivery catheter lumen is configured to retain the expandable segment in the collapsed configuration during delivery; transitioning the expandable segment from the collapsed configuration to the expanded configuration to dilate the vessel and re-establish a channel of blood flow; collapsing the expandable segment; and removing the first blood vessel dilating device from the patient's body.

In some embodiments, the method further includes selecting at least a second blood vessel dilation device including elongated microwire having an expandable segment comprising a pre-set outer diameter greater than the pre-set outer diameter of the expandable segment of the first blood vessel dilating device and substantially equal to or less than the inner diameter of the vessel; delivering the second blood vessel dilation device through the delivery catheter lumen to the affected portion of the vessel; transitioning the expandable segment from the collapsed configuration to the expanded configuration to further dilate the vessel; collapsing the expandable segment from the expanded configuration to the collapsed configuration; and removing the second blood vessel dilating device from the patient's body.

In some embodiments, according to the present invention, the expandable segment further includes a tether coupled to a distal end of the expandable segment, wherein the step of transitioning the expandable segment from the collapsed configuration to the expanded configuration comprises pulling the tether in a proximal direction. In certain embodiments, the method includes expanding the expandable segment to a first pre-set outer diameter by pulling the tether a first predetermined distance in a proximal direction. In further embodiments, the method includes expanding the expandable segment to a second pre-set outer diameter by pulling the tether a second predetermined distance in a proximal direction.

According to other embodiments, the present invention is a device for re-establishing blood flow in a blood vessel including: an elongated microwire having a proximal end and a distal end and including a lumen extending from the proximal end to the distal end and an expandable segment configured to transition from a collapsed configuration to an expanded configuration, the expandable segment comprising at least two struts having a configuration allowing blood flow through the vessel extending from a proximal end to a distal end of the expandable segment and a maximum pre-set outer diameter substantially equal to or less than an inner diameter of the vessel; and a tether coupled to the distal end of the expandable segment and extending within the lumen to a distance beyond the proximal end of the microwire. The blood vessel dilation device can be deliverable through a catheter.

In some embodiments, the expandable segment and the tether are configured such that when the tether is pulled a distance in a proximal direction, the outer diameter of the expandable segment increases by a proportional amount.

According to some embodiments, pulling the tether in a proximal direction transitions the expanded segment from the collapsed configuration to the expanded configuration.

According to other embodiments, the expandable segment and the tether are configured such that when the tether is pulled a predetermined distance in a proximal direction the expandable segment expands to a corresponding pre-set outer diameter.

According to some embodiments, the present invention provides a kit for re-establishing a blood flow channel in a blocked or constricted vessel including a delivery catheter; at least a first blood vessel dilation device including an elongated microwire having a expandable segment configured to transition from a collapsed configuration to an expanded configuration, the expandable segment comprising at least two struts having a configuration allowing blood flow through the vessel and a pre-set outer diameter substantially equal to or less than an inner diameter of the vessel; and at least one additional blood vessel dilation device including an elongated microwire having an expandable segment comprising a pre-set outer diameter greater than the pre-set outer diameter of the expandable segment of the first blood vessel dilating device and substantially equal to or less than the inner diameter of the vessel. Additional blood vessel dilation devices may be provided in the kit. Each additional blood vessel dilation device includes an expandable segment having a pre-set outer diameter greater than the previous device and substantially equal to or less than an inner diameter of the vessel.

### BRIEF DESCRIPTION OF THE SEVERAL VIEWS OF THE DRAWINGS

FIG. 1A is a schematic view of a blood vessel dilation device located within a constricted intracranial vessel in accordance with an embodiment of the present invention.

FIG. 1B is a schematic view of a blood vessel dilation device located within a blocked blood vessel in accordance with an embodiment of the present invention.

FIGS. 2A and 2B are schematic views of a blood vessel dilation device in accordance with an embodiment of the present invention.

FIGS. 3A-3D are schematic views of the expandable segment of the blood vessel dilation device provided in accordance with various embodiments of the present invention.

FIGS. 4A-4B are schematic views showing the delivery of a blood vessel dilation device within a constricted portion of a vessel provided in accordance with various embodiments of the present invention.

FIGS. 5A-5C are schematic views of blood a vessel dilation device including an expandable segment provided in accordance with other embodiments of the present invention.

FIG. 6 is a schematic view of a proximal portion of a blood vessel dilation device provided in accordance with an embodiment of the present invention.

FIG. 7 is a partial, cut-away view of an adjustment feature provided in accordance of an embodiment of the present invention.

FIG. 8 is a schematic view of an adjustment feature provided in accordance with another embodiment of the present invention.

### DETAILED DESCRIPTION OF THE INVENTION

While the embodiments described herein generally refer to use of the blood vessel dilation device in a constricted portion of a cerebral blood vessel, any blocked or constricted vessel is a potential site for treatment. The term “vessel” includes all veins and arteries of the circulatory system. Additionally, the term “vessel” includes various structures of the lymphatic system, including lymph nodes, ducts, capillaries, and vessels. Likewise, as used herein, the term “vessel” also includes the various tube-like structures of the gastrointestinal system.

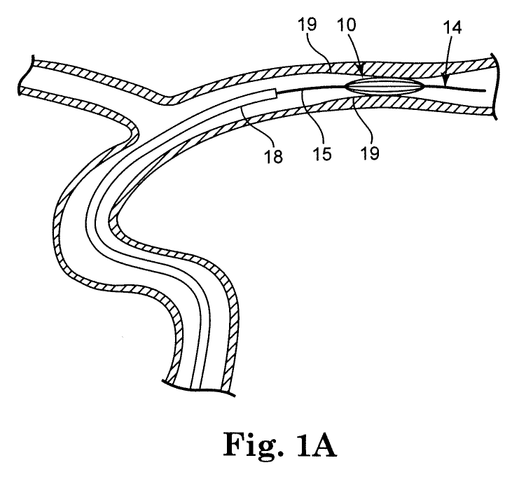

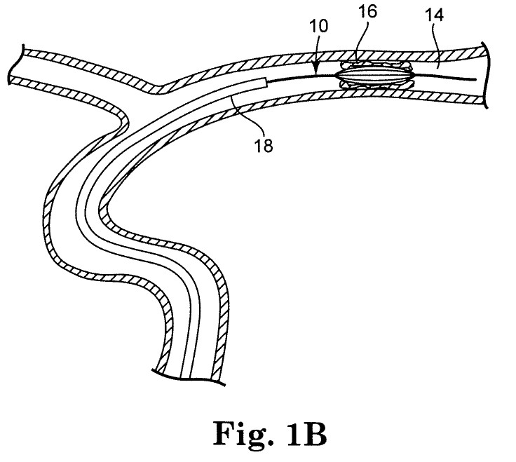

FIG. 1A is a schematic view of a blood vessel dilation device 10 located within a constricted cerebral vessel 14 according to an illustrative embodiment. As shown in FIG. 1A, the blood vessel dilation device 10 can be delivered within the constricted vessel 14 through a delivery catheter 18 using delivery techniques well known to those of skill in the art. According to various embodiments, the blood vessel dilation device 10 is configured to transition from a collapsed configuration to an expanded configuration. FIG. 1A shows the blood vessel dilation device in the expanded configuration. In the expanded configuration, the blood vessel dilation device 10 can dilate the constricted vessel 14 by applying a minimal radial force to the inner walls 19 of the constricted vessel 14. In some embodiments, as will be discussed in further detail below, a series of blood vessel dilation devices 10 can be used in order of increasing outer diameter to dilate the constricted vessel 14 until adequate blood flow through the vessel 14 is restored. Once adequate blood flow through the constricted vessel 14 has been restored, the device 10 is removed from the patient's body.

FIG. 1B is a schematic view of a the device 10 inserted within a blocked portion of a vessel 14. Expansion of the device 10 within the blockage re-establishes a channel of blood flow through the vessel and around the site of the blockage. As shown in FIG. 1B, the blockage is a blood clot 16. Expansion of the device 10 within the clot may cause the clot to break-up. Some portions of the clot 16 may become trapped within the device 10 when the device is collapsed and are removed when the device 10 is removed from the patient's body.

According to various embodiments, the catheter 18 used to deliver the device 10 is small in diameter and has a sufficient length and flexibility such that it can navigate the tortuous pathways of a patient's cerebral vessels to access the blocked or constricted vessel 14. In some embodiments, the catheter 18 is a micro-catheter having an inner diameter of less than about 0.1 cm. In certain embodiments, the catheter 18 may include one or more lumens. For example, the catheter 18 may include a lumen for over the wire-delivery using a guidewire or for the insertion of a stylet. Additionally, the delivery catheter 18 is capable of retaining the expandable segment 30 of the blood vessel dilation device 10 in a collapsed configuration during delivery to the blocked or constricted portion of the vessel 14. According to some embodiments, the catheter 18 can be configured for rapid exchange of a series of blood vessel dilation devices and other devices deliverable through the microcatheter 18.

The catheter 18 can be fabricated from any biocompatible material. For example, the catheter can be fabricated from biocompatible polymers including, but not limited to, the following: polyethylene and copolymers thereof, polyethylene terephthalate or copolymers thereof, nylon, silicone, polyurethanes, fluoropolymers, poly (vinylchloride), and combinations thereof. In further embodiments, a lubricious coating may be provided over the outer surface of the delivery catheter to facilitate delivery through the patient's vasculature system.

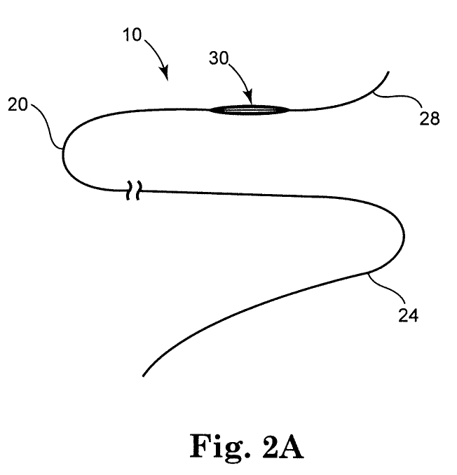

FIGS. 2A and 2B are schematic views of the blood vessel dilation device 10, as shown in FIG. 1, according to illustrative embodiments of the present invention. FIG. 2A shows the blood vessel dilation device 10 in a collapsed configuration suitable for delivery. FIG. 2B shows the blood vessel dilation device 10 in an expandable configuration suitable for dilating a blocked or constricted vessel. As shown in FIGS. 2A and 2B, the blood vessel dilation device 10 includes an elongated microwire 20 having a proximal portion 24, a distal portion 28, and an expandable segment 30. According to various embodiments, the microwire 20 is of sufficient length and flexibility such that it can navigate the tortuous pathways of a patient's cerebral vessels to access the constricted vessel 14. In some embodiments, the microwire 20 is configured to be deliverable through a microcatheter 18 (FIG. 1).

The elongated microwire 20 may be made from any biocompatible material including, but not limited to, stainless steel and any of its alloys; titanium alloys, e.g., nickel-titanium alloys; other shape memory alloys; tantalum; polymers, e.g., polyethylene and copolymers thereof, polyethylene terephthalate or copolymers thereof, nylon, silicone, polyurethanes, fluoropolymers, poly(vinylchloride), and combinations thereof. The diameter of the microwire 20 should be such that the microwire 20 is able to be inserted into and delivered within the narrow vessels of a patient's neurovascular system. According to one embodiment, the diameter of the microwire 20 may range from about 0.013 cm to about 0.13 cm (about 0.005 inches to about 0.05 inches). According to another embodiment, the diameter of the microwire 20 may range from about 0.013 cm to about 0.076 cm (about 0.005 inches to about 0.03 inches). According to yet another embodiment, the diameter of the microwire 20 may range from about 0.015 cm to about 0.030 cm (about 0.006 inches to about 0.012 inches).

The expandable segment 30 can be coupled to or formed integrally with the distal portion 28 of the elongated microwire 20. Important physical characteristics of the expandable segment 30 include, but are not limited to: length, pre-set outer diameter in the expanded configuration, degree of flexibility and lateral stiffness, the amount of radial force involved when transitioning from a collapsed configuration to an expanded configuration, automatic expansion or controlled expansion, and the like. These physical properties can be modified to account for such factors as the inner diameter of the constricted vessel, length of the constricted portion of the vessel, or type of luminal structure (e.g., artery or vein) affected by vasospasm. Additionally, the open configuration of the expandable segment 30 allows for blood flow through the vessel 14.

In some embodiments, the expandable segment 30 is configured to automatically expand from a collapsed configuration, as shown in FIG. 2A, for insertion into the constricted vessel 14 to an expanded configuration, as shown in FIG. 2B, having a pre-set outer diameter. Limiting the expansion of the expandable segment 30 to a pre-set outer diameter may prevent over expansion of the expandable segment 30 with respect to the inner diameter of the constricted vessel 14 which may result in procedural complications. The pre-set outer diameter is selected such that it is substantially equal to or less than the inner diameter of the vessel 14 into which the expandable segment 30 is inserted.

The pre-set diameter of the expanded segment can vary depending on the severity of the constriction, the type and/or size of the constricted vessel, the location of the constricted vessel within the patient's body, and other factors. In one embodiment, the pre-set outer diameter of the expandable segment 30 can range from about 0.25 mm to 5 mm. According, to another embodiment, the pre-set outer diameter can range from about 0.5 mm to about 2.5 mm. Additionally, the length of the expandable segment 30, when expanded, should be sufficient to treat the entire length of the constricted portion of the affected vessel. According to various embodiments, the length of the expanded segment 30 is at least 3 mm when expanded. According to another embodiment, the length of the expanded segment 30 ranges from about 3 mm to about 20 mm.

Additionally, the pre-set outer diameter is selected such that, when expanded, the expandable segment 30 contacts the inner walls 19 of the constricted vessel 14 with a minimal amount of radial force to gently push the inner vessel walls 19 to open, resulting in an increase in the inner diameter of the vessel 14. Limiting the radial expansive force may prevent trauma to the vessel 14. According to various embodiments, the expandable segment 30 is configured such that it applies a sufficient amount of radial expansion force to the inner walls 19 of the vessel without inducing trauma or rupture to the vessel 14. According to one embodiment, the expandable segment 30 is configured to provide a normalized radial force of about 18 to about 19 grams/mm of length. In some embodiments, the expandable segment 30 is configured to place a minimal amount of radial force on the inner walls of the constricted vessel such that the inner diameter of the vessel 14 increases by a sufficient amount to re-establish a blood flow channel. In some embodiments, the expandable segment 30, when expanded, increases the inner diameter of the vessel 14 by at least 10%-20% and more preferably, 20-50%.

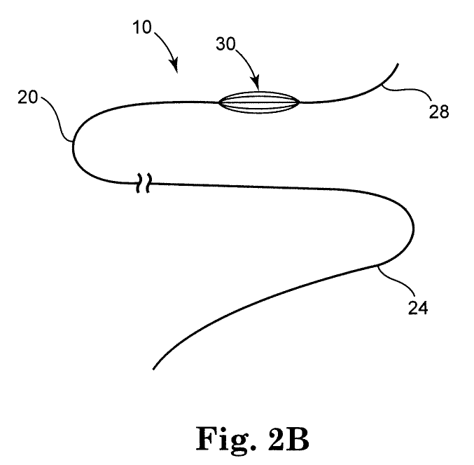

FIGS. 3A to 3D are schematic views of the expandable segment 30 according to various embodiments of the present invention. According to various embodiments, the expandable segment 30 is fabricated from a shape memory material. Exemplary shape memory materials include Nitinol, MP35N, and other materials well-known in the art. According to one embodiment, the expandable segment 30 can be formed from a laser-cut Nitinol tube using techniques generally known in the art. The Nitinol tube is cut with a laser to remove material leaving behind at least one collar having a diameter equal to that of the original tube diameter and one or more integrally formed, expandable struts 34, as shown in FIGS. 3A and 3B. In certain embodiments, the struts 34 extend from a proximal end 36 to a distal end 38 of the expandable segment and have a configuration that allows blood flow through the vessel. Additionally, the struts 34 are spaced an equal distance from one another such that the force is equally applied around an inner circumference of the inner walls of the vessel. The number of struts 34 can vary. For example, in some embodiments the number of struts 34 can range from about 2 to about 8. In other embodiments, the number of struts 34 can range from about 3 to about 5. In some embodiments, the struts 34 can be connected to form one or more cells, as shown in FIG. 3C. As the number of cells increase, the expandable segment 30 can take on a cage or basket-like appearance. According to various other embodiments, the struts 34 can be spiraled, canted or arced and can have various configurations adapted to minimize the amount of strain on the struts 34 and the amount of force placed on the vessel walls. In one embodiment, as shown in FIG. 3D, the struts 34 have a spiraled configuration. In general, the expandable struts 34 may have any configuration capable of transitioning from a collapsed configuration to an expanded configuration, wherein in the expanded configuration the struts place a minimal amount of a radial force to the inner walls of the blocked or constricted vessel 14 to dilate the vessel 14.

In certain embodiments, the exterior surface of the expandable segment 30 may be configured to prevent the activation of pathological processes during or after implant deployment. For example, the exterior surface of the expandable segment 30 may be formed to be smooth to decrease the likelihood of damage upon expansion of the expandable segment which may prevent an inflammatory response.

In some embodiments, the expandable segment 30 may also include a therapeutic agent. The therapeutic agent may be coated onto the implant, mixed with a biodegradable polymer or other suitable temporary carrier and then coated onto the implant, or, when the implant is made from a polymeric material, dispersed throughout the polymer. Exemplary therapeutic agents include, but are not limited to, the following: antibiotics, anticoagulants, antifungal agents, anti-inflammatory agents, antineoplastic agents, antithrombotic agents, endothelialization promoting agents, free radical scavengers, immunosuppressive agents, thrombolytic agents, vasodilating agents, and any combination thereof. In one embodiment, the therapeutic agent is an anti-inflammatory agent. In other embodiments, the therapeutic agent is a vasodilating agent. Additionally, in some embodiments the microwire and/or the expandable segment 30 can include a radiopaque marker or coating for visualization purposes.

In some embodiments, the microwire 20 and/or the expandable segment 30 may include a lubricious coating to facilitate advancement of the device 10 through a patient's neurovasculature system. The lubricious coating may include hydrophilic polymers such as polyvinylpyrrolidone-based compositions, fluoropolymers such as tetrafluoroethylene, or silicones. In one embodiment, the lubricious coating may include a hydrophilic coating or gel.

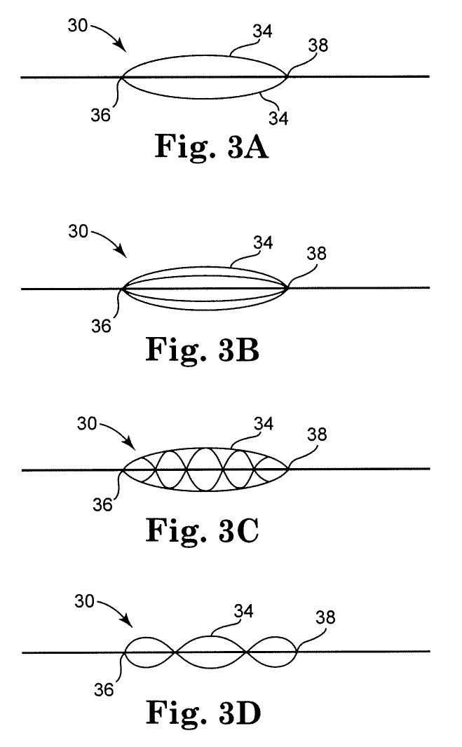

FIGS. 4A and 4B are schematic views showing delivery of the blood vessel dilation device 10 within a blocked or constricted portion 38 of an affected vessel 14 using the delivery catheter 18 according to an embodiment of the present invention. As shown in FIG. 4A, the distal portion 28 of the blood vessel dilation device 10 including the expandable segment 30 is contained within the delivery catheter 18 for insertion within the blocked or constricted vessel. FIG. 4B shows the expandable segment 30 in the expanded configuration after it has been deployed from the catheter 18. The blood vessel dilation device 10 is selected based on the pre-set outer diameter of the expandable segment 30 and the size and location of the affected vessel 14. According to some embodiments, the expanded segment 30 is selected such that it has a pre-set outer diameter substantially equal to or less than an inner diameter of the affected vessel 14 when the vessel is not blocked or constricted. In other embodiments, the expandable segment 34 can be selected such that it has a pre-set outer diameter equal to or slightly less than the estimated inner diameter of the blocked or constricted portion of the vessel 14. Additionally, the expanded segment 30 may be selected such that is has an expanded length substantially equal to a length of the blocked or constricted portion 36 of the vessel 14. If the blocked or constricted portion of the vessel 14 is particularly narrow, an initial blood vessel dilation device 10 having a very small pre-set outer diameter may be initially selected.

According to various embodiments of the present invention, the expandable segment 30 can be transitioned from a collapsed configuration, as shown in FIG. 4A to an expanded configuration, as shown in FIG. 4B, using the delivery catheter 18. First, the delivery catheter 18 including the blood vessel dilation device 10 is guided to and positioned within the blocked or constricted portion 38 of the affected vessel 14 using delivery techniques known to those of skill in the art. The delivery catheter 18 and blood vessel dilation device 10 is positioned within the blocked or constricted portion 38 of the vessel 14 such that when expanded, the expandable member 30 will extend within the entire length of the blocked or constricted portion. Next, according to one embodiment, the blood vessel dilation device 10 is held in a fixed position relative to the catheter 18, and the catheter 18 is moved in a proximal direction relative to the blood vessel dilation device 10 to transition the expandable segment 30 from the collapsed configuration to an expanded configuration. In another embodiment, the catheter 18 is held in a fixed position relative to the blood vessel dilation device 10, and the blood vessel dilation device 10 is then moved in a distal direction relative to the catheter 18 to transition the expandable segment 30 to the expanded configuration.

Depending upon the inner diameter of the blocked or constricted portion 36 of the vessel 14, the expandable segment 30 expands to a diameter equal to or less than its pre-set diameter placing a minimal amount of a radial expansion force to the inner vessel walls 19. The inner vessel walls 19 are dilated by the continual expansion of the expandable segment 30 until the expandable segment has reached its pre-set diameter. During the expansion process, blood flow through the blocked or constricted vessel 14 is monitored and evaluated to determine if adequate blood flow through the vessel has been re-established. If dilation of the blocked or constricted portion 38 of the vessel 14 is insufficient to restore adequate blood flow, the blood vessel dilation device 10 is withdrawn from the patient's body through delivery catheter 18, and the process is repeated using a blood vessel dilation device 10 including an expandable segment 30 having a larger pre-set outer diameter. According to certain embodiments, these successive steps can be performed in rapid succession. According to some embodiments, the process can be repeated using a series of blood vessel dilation devices 10 each having an expandable segment 30 of an increasing pre-set outer diameter until adequate blood flow through the vessel is restored. After blood flow through the vessel 14 has been restored, the final blood vessel dilation device 10 is removed from the patient's body.

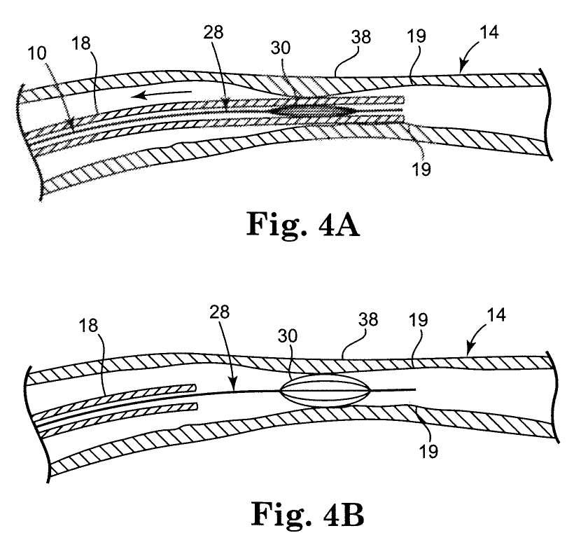

FIG. 5A-FIG. 5C are schematic views of a blood vessel dilation device 100 according to another embodiment of the present invention. As shown in FIGS. 5A-5C, the blood vessel dilation device 100 includes an elongated microwire 120 having a proximal portion 124, a distal portion 128, an expandable segment 130, and a distal tip segment 131. In some embodiments, the distal tip segment 131 is floppy to facilitate navigation through the tortuous pathways of the vascular system in the absence of a delivery catheter or guidewire and to prevent damage to the internal walls of the vasculature. The microwire 120 also includes at least one lumen 132. As shown in FIGS. 5A-5C, a tether 134 is coupled to a distal end 136 of the expandable segment 130 and extends within the lumen 132. According to some embodiments, the tether 132 extends within the lumen 132 from the distal end 136 of the expandable segment 130 to a distance beyond a proximal end 138 of the microwire 120, and is accessible to the clinician.

In some embodiments, the blood vessel dilation device 100 can be delivered through a catheter such as catheter 18, described above. According to other embodiments, the blood vessel dilation device 100 can include a lumen and can be delivered using over the wire delivery techniques. In yet other embodiments, the device 100 can be delivered without the aid of a delivery member such as a catheter or guidewire.

According to various embodiments, expandable segment 130 is configured to controllably expand from a collapsed configuration as shown in FIG. 5A to an expanded configuration, as shown in either FIG. 5B or 5C. Expansion and collapse of the expandable segment 130 is controlled by the tether 134. For example, when tension is applied to the tether 134 by pulling the tether 134 in a proximal direction as indicated by the arrows in FIGS. 5A-5C, the expandable segment 130 transitions from a collapsed configuration (FIG. 5A) to an expanded configuration (FIG. 5B or FIG. 5C).

The amount of expansion and the rate at which the expandable segment 130 is expanded can be controlled by the clinician. To prevent over-expansion, the expandable segment has a maximum pre-set outer diameter. According to some embodiments, the maximum pre-set outer diameter of the expandable segment is substantially equal to or less than an inner diameter of the vessel 14, when not subject to vasospasm. The type and location of the blocked or constricted vessel 14 are taken into account when selecting the appropriate blood vessel dilation device 100 to be used in the procedure. According to some embodiments, the expandable segment 130 and tether 134 and are configured such that when the tether 134 is pulled a specified distance in a proximal direction, the outer diameter of the expandable segment increases by a proportional amount. For example, when the tether 134 is pulled a distance of 1 mm in the distal direction, the outer diameter of the expandable segment 130 increases by 1 mm. According to another embodiment, the expandable segment 130 and tether 134 are configured such that when the tether 130 is pulled a specified distance in a proximal direction, the expandable segment 130 expands to a pre-set outer diameter. Expansion of the expandable segment 130 can be continued in small increments until blood flow through the blocked or constricted portion of the vessel has been restored. A blood vessel dilation device 100 having this configuration allows for a single device to be used to dilate the vessel, eliminating a need for a series of devices. The expandable segment 130 can be transitioned from the expanded configuration to the collapsed configuration by the release of tension on the tether 134. Once the expanded segment is in the collapsed configuration, the blood vessel dilation device 100 can be withdrawn from the patient's body.

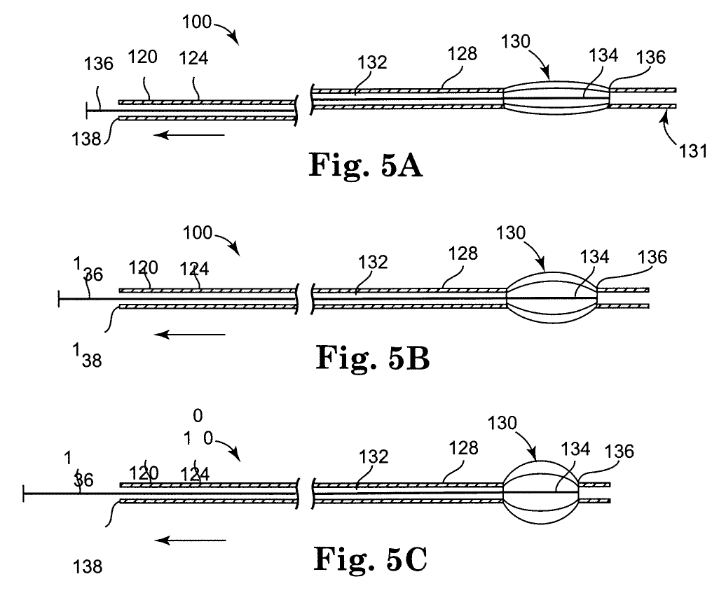

FIG. 6 is a schematic view of a proximal portion 124 of the blood vessel dilation device 100, shown in FIGS. 5A-5C. As shown in FIG. 6, the proximal portion 124 includes a handle portion 140 having an incremental adjustment feature 150. The adjustment feature 150 is operably coupled to the tether 136 and when manipulated, manipulates the tether 136 in a proximal direction to controllably actuate the expandable portion 130 (not shown). The adjustment feature 150 is calibrated to precisely expand the expandable portion 130 by incremental amounts.

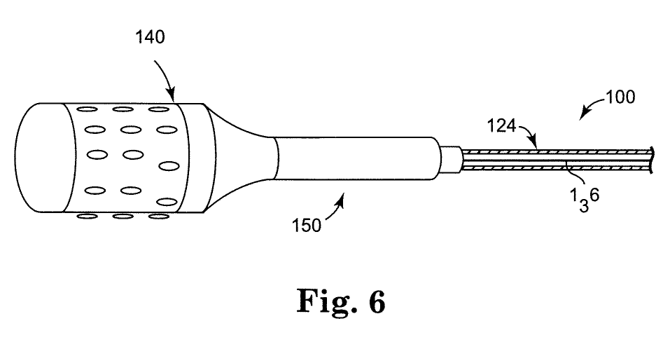

FIG. 7 is a schematic view of the adjustment feature 150 coupled to the tether 136 according to one embodiment of the present invention. As shown in FIG. 7, the adjustment feature 150 includes a slot 154 having a plurality of ridges, teeth, or other cooperating structures 156 configured to cooperate with a post 158 or other structure coupled to the tether 136 to manipulate the tether 136 in a proximal or distal direction as indicated by the arrow. The ridges or teeth 156 are spaced an equal distance from each other and are calibrated such that when the post 158 is moved in a proximal direction to a space 160 existing between the next set of teeth 156, the expandable portion 130 is expanded by a proportional amount. For example, in one embodiment, the teeth 156 can be spaced about 0.5 mm apart such that when the post 156 is manipulated in a proximal direction from a first space 160 between a first set of teeth 156 to a second space 160 between the next set of teeth 156, this results in a 0.5 mm expansion of the expandable portion 130. This arrangement facilitates precise and repeatable expansion and collapse of the expandable portion 130. In further embodiments, the device 100 may make an audible “clicking” sound when the post 136 is moved in the slot 154.

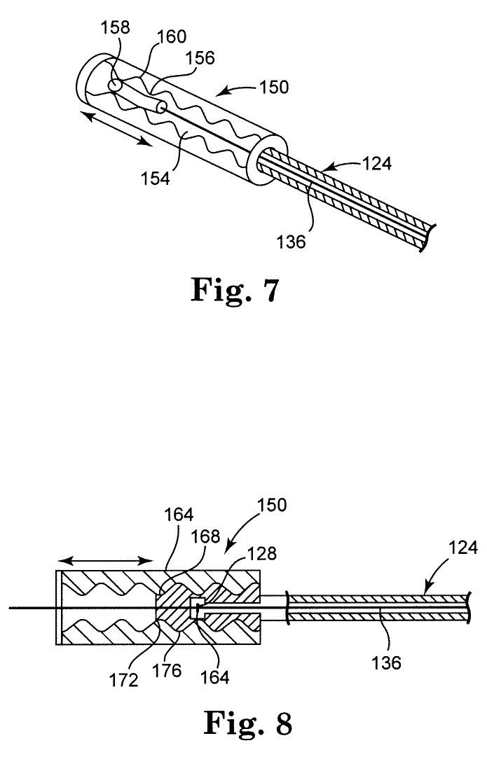

FIG. 8 is a partial cut-away, schematic view of the incremental adjustment feature 150 according to another embodiment of the present invention. The adjustment feature includes an outer portion 164 including internal threads 168 threadably engaged with an inner portion 172 having external threads 176. As shown in FIG. 8, a proximal end 178 of the tether 136 terminates within and is coupled to the inner portion 172. Rotation of the outer portion 164 relative to the inner portion 172 results in proximal or distal movement of the tether 136. In some embodiments, the threads 168 and 176 on the inner and outer portions 164 and 172, respectively, can be calibrated such that one full rotation of the outer portion 164 results in a one full rotation of the inner portion 172.

The threads 168 and 176 are spaced an equal distance from each other such that a rotation of the outer portion 164 results in an incremental adjustment of the tether 136. For example, in one embodiment, if the threads 168 and 176 are spaced about 1 mm from each other, one full rotation of the outer portion 164 results in the tether 136 moving a distance about 1 mm resulting in a 1 mm expansion of the expandable portion 130. In some embodiments, the adjustment feature 150 can be calibrated such that the expandable portion expands by about less than 2 mm, less than 1 mm, and in some embodiments less than about 0.5 mm. The incremental adjustment feature 150 facilitates precise, controllable expansion and collapse of the expandable portion 130. In some embodiments, the incremental adjustment feature can be calibrated such that it controls the expansion of the expandable portion 130 within less than 0.5 mm and more preferably, within less than 0.1 mm.

In further embodiments, the adjustment feature 150 can also include a locking feature for securing the tether 136 at a desired position to prevent over expansion or unintentional collapse of the expandable portion 130. In one further embodiment, the locking feature can include an external nut that when engaged with the adjustment feature 150 is configured to apply a frictional force to the proximal end 178 of the tether 136 to prevent further movement of the tether 136 in either a proximal or distal direction. In another embodiment, the locking feature can include a pair of cooperating jaws that when engaged, clamp down on the proximal end of 178 of the tether 136 to prevent further movement.

Patents and patent applications disclosed herein, including those cited in the Background of the Invention, are hereby incorporated by reference. Other embodiments of the invention are possible. Although the description above contains many specificities, these should not be construed as limiting the scope of the invention, but as merely providing illustrations of some of the presently preferred embodiments of this invention. Thus the scope of this invention should be determined by the appended claims and their legal equivalents. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims.

What is claimed is:

1. A method for re-establishing a blood flow channel in a blocked or constricted portion of an affected vessel within a patient's body, the method comprising:
selecting at least a first blood vessel dilation device including an elongated microwire having a expandable segment configured to transition from a collapsed configuration to an expanded configuration, the expandable segment comprising at least two struts having a configuration allowing blood flow through the vessel and a pre-set outer diameter substantially equal to or less than an inner diameter of the vessel;
advancing a delivery catheter including at least one lumen to a position within the blocked or constricted portion of the vessel;
delivering the first blood vessel dilation device through the delivery catheter lumen to the blocked or constricted portion of the vessel, wherein the delivery catheter lumen is configured to retain the expandable segment in the collapsed configuration during delivery;
transitioning the expandable segment from the collapsed configuration to the expanded configuration to dilate the blocked or constricted portion of the affected vessel;
evaluating blood flow through the vessel;
collapsing the expandable segment; and
removing the first blood vessel dilating device from the patient's body.

2. The method according to claim 1, further comprising dilating the blocked or constricted portion of the vessel by an amount sufficient to re-establish a blood flow channel in the vessel.

3. The method according to claim 1, further comprising dilating the blocked or constricted portion of the blood vessel by at least 10%.

4. The method according to claim 1, wherein the step of transitioning the expandable segment from the collapsed configuration to the expanded configuration comprises moving the catheter in a proximal direction relative to the blood vessel dilation device which is held in a fixed position relative to the catheter.

5. The method according to claim 1, wherein the step of transitioning the expandable segment from the collapsed configuration to the expanded configuration comprises moving the blood vessel dilation device in a distal direction relative to the catheter which is held in a fixed position.

6. The method according to claim 1, wherein the step of collapsing the expandable segment comprises retracting the expandable segment into the delivery catheter, wherein the catheter is held in a fixed position relative to the blood vessel dilation device.

7. The method according to claim 1, wherein the step of collapsing the expandable segment comprises holding the blood vessel dilation device including the expandable segment in a fixed position and moving the catheter in a distal direction relative to the blood vessel dilation device such that the expandable segment transitions from the expanded configuration to the collapsed configuration and is retained in the collapsed configuration within the catheter.

8. The method according to claim 1, further comprising:
selecting at least a second blood vessel dilation device including elongated microwire having an expandable segment comprising a pre-set outer diameter greater than the pre-set outer diameter of the expandable segment of the first blood vessel dilating device and substantially equal to or less than the inner diameter of the vessel;
delivering the second blood vessel dilation device through the delivery catheter lumen to the blocked or constricted portion of the vessel;
transitioning the expandable segment from the collapsed configuration to the expanded configuration to further dilate the blocked or constricted portion of the vessel;
collapsing the expandable segment from the expanded configuration to the collapsed configuration;
evaluating blood flow through the vessel; and
removing the second blood vessel dilating device from the patient's body.

9. The method according to claim 1, wherein the expandable segment further comprises a tether coupled to a distal end of the expandable segment, and wherein the step of transitioning the expandable segment from the collapsed configuration to the expanded configuration comprises manipulating the tether in a proximal direction.

10. The method according to claim 9, wherein manipulating the tether coupled to a distal end of the expandable portion comprises moving the tether by a specified distance in the proximal direction to controllably expand the expandable portion by a proportional amount.

11. The method according to claim 9, wherein the step of collapsing the expandable segment comprises releasing the tether to transition the expandable segment from the expanded configuration to the collapsed configuration for removal from the patient's body.

12. The method according to claim 9, further comprising expanding the expandable segment to a first pre-set outer diameter by moving the tether a first predetermined distance in a proximal direction.

13. The method according to claim 9, further comprising expanding the expandable segment to a second pre-set outer diameter by moving the tether a second predetermined distance in a proximal direction.

14. A method of dilating a blocked or constricted portion of an affected vessel within a patient's body, the method comprising:
delivering a blood vessel dilation device to the blocked or constricted portion of the blood vessel, the blood vessel dilation device comprising an elongated microwire including an expandable segment having a distal end and comprising at least two struts having a configuration for allowing blood flow through the vessel, and a tether coupled to the distal end of the expandable segment;
moving the tether at least a first pre-determined distance in a proximal direction to expand the expandable segment to a first pre-set outer diameter to dilate the blocked or constricted portion of the vessel; and
evaluating blood flow through the vessel.

15. The method according to claim 14, further comprising moving the tether at least a second predetermined distance in a proximal direction to expand the expandable segment to a second pre-set outer diameter to further dilate the blocked or constricted portion of the vessel.

16. The method according to claim 14, further comprising releasing the tether to collapse the expandable segment.

17. The method according to claim 14, further comprising removing the blood vessel dilation from the patient's body.

18. The method according to claim 14, wherein the expandable segment comprises a maximum pre-set outer diameter substantially equal to or less than an inner diameter of the vessel.

19. A blood vessel dilation device for dilating a blocked or constricted portion of a vessel in a patient's body, the device comprising:
an elongated microwire having a proximal end and a distal end and including a lumen extending from the proximal end to the distal end and an expandable segment configured to transition from a collapsed configuration to an expanded configuration, the expandable segment comprising at least two struts having a configuration allowing blood flow through the vessel extending from a proximal end to a distal end of the expandable segment and a maximum pre-set outer diameter substantially equal to or less than an inner diameter of the vessel;
a tether coupled to the distal end of the expandable segment and extending within the lumen to a distance beyond the proximal end of the microwire; and
a handle portion coupled to the proximal end of the microwire and including an incremental adjustment feature coupled to a proximal end of the tether for controllably expanding the expandable segment.

20. The device according to claim 19, wherein the adjustment feature includes a slot having a plurality of equally spaced teeth configured to cooperate with a post coupled to the proximal end of the tether.

21. The device according to claim 19, wherein the adjustment feature includes an outer portion having threadably engaged with an inner portion coupled to the proximal end of the tether, wherein rotation of the outer portion results in movement of the tether in a proximal or distal direction.

22. The device according to claim 19, wherein the adjustment feature further comprises a locking feature for securing the proximal end of the tether at a desired position.

23. The blood vessel dilation device according to claim 19, wherein the expandable segment and the tether are configured such that when the tether is moved a specified distance in a proximal direction, the outer diameter of the expandable segment increases by a proportional amount.

24. The blood vessel dilation device according to claim 19, wherein moving the tether in a proximal direction transitions the expanded segment from the collapsed configuration to the expanded configuration.

25. The blood vessel dilation device according to claim 19, wherein the expandable segment and the tether are configured such that when the tether is moved a predetermined distance in a proximal direction the expandable segment expands to a corresponding pre-set outer diameter.

26. The blood vessel dilation device according to claim 19, wherein the device is deliverable through a delivery catheter.
